(19) United States
(12) Patent Application Publication
(10) Pub. No.: US 2021/0038191 A1
(43) Pub. Date: Feb. 11, 2021

(54) METHODS AND SYSTEMS FOR PROLONGING BATTERY LIFE OF ULTRASOUND DEVICES

(71) Applicant: Butterfly Network, Inc., Guilford, CT (US)

(72) Inventors: Matthew R. Hageman, Winfield, IL (US); Robert Schneider, Killingworth, CT (US); John Peabody, Guilford, CT (US)

(21) Appl. No.: 16/988,057

(22) Filed: Aug. 7, 2020

Related U.S. Application Data
(60) Provisional application No. 62/885,168, filed on Aug. 9, 2019.

(51) Int. Cl. A61B 8/00; G01S 7/52

(52) U.S. Cl. CPC A61B 8/4427; A61B 2560/0214; A61B 8/565; G01S 7/52096

(57) ABSTRACT

Aspects of the technology described herein relate to prolonging battery life in an electronic device, such as ultrasound devices. A system may include an ultrasound device and a package. The ultrasound device may include magnetic sensing circuitry. The package may include a lid, and the lid may include a magnet. The magnetic sensing circuitry may control power received by communication circuitry in the ultrasound device based on detecting a magnet that is external to the ultrasound device. For example, the magnetic sensing circuitry may disable power rails in the power circuitry that provide power to communication circuitry when the magnetic sensing circuitry detects the magnet. When the magnetic sensing circuitry cannot detect the magnet, the power circuitry may enter a default state in which power rails provide power to the communication circuitry. The ultrasound device may include an indicator that enters a turn-on sequence when the lid is removed from the package.

### CROSS-REFERENCE TO RELATED APPLICATIONS

This application claims the benefit under 35 U.S.C. § 119(e) of U.S. Patent Application Ser. No. 62/885,168, filed Aug. 9, 2019 under Attorney Docket No. B1348.70153US00, and entitled “METHODS AND SYSTEMS FOR PROLONGING BATTERY LIFE,” which is hereby incorporated by reference herein in its entirety.

### FIELD

Generally, the aspects of the technology described herein relate to methods and systems for prolonging battery life, including battery life of ultrasound devices.

### BACKGROUND

An electronic device may include a battery that provides power to the electronic device. Certain subsystems in the electronic device may draw current from the battery, thereby reducing the battery life of the battery.

### SUMMARY

According to one aspect, a system comprises an electronic device, such as an ultrasound device, comprising magnetic sensing circuitry configured to control power received by a portion of the electronic device based on detecting a magnet that is external to the electronic device.

In some embodiments, the electronic device further comprises power circuitry, and wherein the magnetic sensing circuitry is configured to control the power circuitry based on detecting the magnet. In some embodiments, the magnetic sensing circuitry is configured to disable one or more power rails in the power circuitry that provide power to the portion of the electronic device based on detecting the magnet. In some embodiments, the magnetic sensing circuitry is configured to disable one or more power rails in the power circuitry when the magnetic sensing circuitry detects the magnet. In some embodiments, the electronic device is configured such that, when the magnetic sensing circuitry cannot detect the magnet, the power circuitry is configured to enter a default state in which power rails in the power circuitry provide power to the portion of the electronic device.

In some embodiments, the electronic device further comprises power circuitry, and wherein the magnetic sensing circuitry is configured to decouple the power circuitry from a battery based on detecting the magnet. In some embodiments, the magnetic sensing circuitry is configured to decouple the power circuitry from the battery when the magnetic sensing circuitry detects the magnet. In some embodiments, the electronic device is configured such that, when the magnetic sensing circuitry cannot detect the magnet, the power circuitry is configured to enter a default state in which the power circuitry is coupled to the battery. In some embodiments, the magnetic sensing circuitry comprises a digital-output magnetic sensor.

In some embodiments, the electronic device further comprises communication circuitry, and the portion of the electronic device controlled by the magnetic sensing circuitry comprises the communication circuitry. In some embodiments, the communication circuitry is configured to facilitate communication between the electronic device and a processing device. In some embodiments, the processing devices comprises a mobile phone, a tablet, or a laptop.

In some embodiments, the communication circuitry comprises wired communication circuitry for communicating over a wired communication link. In some embodiments, the communication circuitry comprises wireless communication circuitry for communicating over a wireless communication link.

In some embodiments, the electronic device has a low power state in which the communication circuitry is powered on but other circuitry in the electronic device is powered off, and the communication circuitry is configured, based on detecting connection of a communication link between the electronic device and the processing device, to turn on the other circuitry in the electronic device. In some embodiments, the communication link comprises a cable. In some embodiments, the communication circuitry comprises a Universal Serial Bus (USB) subsystem.

In some embodiments, the electronic device further comprises an indicator, and the indicator is configured to receive or not receive power based on whether the magnetic sensing circuitry detects or does not detect the magnet. In some embodiments, the indicator comprises a light-emitting diode (LED) on an exterior of the electronic device. In some embodiments, the indicator is configured to enter a turn-on sequence upon receiving power.

In some embodiments, the system further comprises a package comprising a lid, the lid comprising a magnet, and the electronic device and the lid are configured such that, when the lid is removed from the package, the indicator enters a turn-on sequence. In some embodiments, the system further comprises a package that comprises the magnet. In some embodiments, the package comprises a lid, and the lid comprises the magnet. In some embodiments, the package comprises a box. In some embodiments, the package comprises a case. In some embodiments, the package is a package in which the electronic device is shipped to a customer. In some embodiments, the package is a package in which the electronic device is stored. In some embodiments, the magnet is disposed in a portion of the package such that, when the electronic device is disposed in the package, the magnet is adjacent to a portion of the electronic device in which the magnetic sensing circuitry is disposed. In some embodiments, the electronic device and the package are configured such that, when the electronic device is in the package, the magnet is sufficiently close to the electronic device such that the magnetic sensing circuitry detects the magnet.

In some embodiments, the magnet is a standalone magnet. In some embodiments, the system further comprises a wand that comprises the magnet. In some embodiments, the electronic device comprises an ultrasound device.

According to another aspect, an electronic device comprises power circuitry to supply power for operation of the electronic device, the power circuitry configured for operable communication with a product package of the electronic device, where the power circuitry automatically places the electronic device in a reduced power consumption state whenever the electronic device is disposed within the product package and the product package is in a closed state, and the power circuitry automatically places the electronic device in a normal power consumption state whenever the product package is in an open state and/or the electronic device is removed from the product package.

Some aspects include a method to provide the systems and/or electronic devices in accordance with the above aspects and embodiments.

### BRIEF DESCRIPTION OF THE DRAWINGS

Various aspects and embodiments will be described with reference to the following exemplary and non-limiting figures. It should be appreciated that the figures are not necessarily drawn to scale. Items appearing in multiple figures are indicated by the same or a similar reference number in all the figures in which they appear.

FIG. 1A is a schematic block diagram of an example system for prolonging battery life in an electronic device, in accordance with certain embodiments described herein;

FIG. 1B is a schematic block diagram of another example system for prolonging battery life in an electronic device, in accordance with certain embodiments described herein;

FIG. 1C is a schematic block diagram of another example system for prolonging battery life in an electronic device, in accordance with certain embodiments described herein;

FIG. 2 is a schematic block diagram of an example electronic device, in accordance with certain embodiments described herein;

FIG. 3 is another schematic block diagram of an example electronic device, in accordance with certain embodiments described herein;

FIG. 4 is another schematic block diagram of an example electronic device, in accordance with certain embodiments described herein;

FIG. 5 is another schematic block diagram of an example electronic device, in accordance with certain embodiments described herein;

FIG. 6 is another schematic block diagram of an example system for prolonging battery life in an electronic device, in accordance with certain embodiments described herein;

FIG. 7A is another schematic block diagram of an example system for prolonging battery life in an electronic device, in accordance with certain embodiments described herein;

FIG. 7B is another schematic block diagram of an example system for prolonging battery life in an electronic device, in accordance with certain embodiments described herein;

FIG. 8 is another schematic block diagram of an example system for prolonging battery life in an electronic device, in accordance with certain embodiments described herein;

FIG. 9 is a perspective view of a system for prolonging battery life, in accordance with certain embodiments described herein;

FIG. 10 is a perspective view of the system for prolonging battery life of FIG. 9 in operation, in accordance with certain embodiments described herein;

FIG. 11 is another perspective view of the system for prolonging battery life of FIG. 9 in operation, illustrating the state of the system after the state of FIG. 10, in accordance with certain embodiments described herein;

FIG. 12 is another perspective view of the system for prolonging battery life of FIG. 9 in operation, illustrating the state of the system after the state of FIG. 11, in accordance with certain embodiments described herein;

FIG. 13 is another perspective view of the system for prolonging battery life of FIG. 9 in operation, illustrating the state of the system after the state of FIG. 12, in accordance with certain embodiments described herein;

FIG. 14 is another perspective view of the system for prolonging battery life of FIG. 9 in operation, illustrating the state of the system after the state of FIG. 13, in accordance with certain embodiments described herein;

FIG. 15 is another perspective view of the system for prolonging battery life of FIG. 9 in operation, illustrating the state of the system after the state of FIG. 14, in accordance with certain embodiments described herein; and

FIG. 16 is another perspective view of the system for prolonging battery life of FIG. 9 in operation, illustrating the state of the system after the state of FIG. 15, in accordance with certain embodiments described herein.

### DETAILED DESCRIPTION

Battery life may be a concern for certain electronic devices. Certain electronic devices may include low power modes in which certain subsystems are powered on. Moreover, some electronic devices may have certain subsystems powered on even when packaged in product containers, residing in storage/inventory, and/or in the process of being shipped to customers. However, the subsystems that are powered on may draw current from the battery of the electronic device when packaged, stored, and/or being shipped, thereby reducing the life of the battery.

The inventors have recognized that it may be helpful to power down these subsystems during such times between final product packaging and when a customer/end user finally accesses the electronic device for use. While one solution could be a power switch that a user activates after removing the electronic device from a package, a power switch may be a potential point of ingress of fluid, and presents the possibility of accidental pressing of the power switch during normal use. The inventors have recognized that magnetic sensing circuitry may be included in an electronic device, where the magnetic sensing circuitry is configured to control power received by a portion (where a portion may include all) of the electronic device. When the magnetic sensing circuitry detects the magnetic field of a nearby magnet, the magnetic sensing circuitry may be configured to control power circuitry or a gate between the battery and the power circuitry to power down certain subsystems in the electronic device. When the magnetic sensing circuitry does not detect the magnetic field of a nearby magnet, the electronic device may be configured to enter a default mode in which certain subsystems are powered on. The inventors have recognized that a magnet may be included in a package in which the electronic device is shipped (e.g., in the lid of the package), such that the magnetic sensing circuitry in the electronic device is sufficiently close to the magnet in the package. The magnetic sensing circuitry may then detect the magnetic field of the magnet in the package and power down the subsystems while the electronic device is in the package. When the magnet is no longer sufficiently close to the magnetic sensing circuitry in the electronic device, such as when the lid that includes the magnet has been removed from the package and/or when the electronic device has been removed from the package, then the subsystems may automatically power on. When the magnetic sensing circuitry keeps the subsystems powered down, current draw from the battery may be reduced, and the life of the battery may be prolonged. Thus, the package may be a package configured to hold an electronic device, where the package is separate from the electronic device, and where the package includes a magnet for prolonging the battery life of the electronic device.

As an example, an electronic device may have a low power mode in which communication circuitry is powered on. When the communication circuitry detects connection of a communication link (e.g., a cable) to the electronic device, the communication circuitry may be configured to turn on the rest of the electronic device. For example, the electronic device may be an ultrasound device, and the communication circuitry may be a Universal Serial Bus (USB) subsystem that turns on the rest of the ultrasound device for imaging upon detecting connection of a USB cable to the ultrasound device (e.g., between the ultrasound device and a processing device such as a smartphone). When the electronic device is in a package containing a magnet, the communication circuitry may be powered down. It may be appropriate to power down the communication circuitry when the electronic device is in the package because the electronic device may not be ready for connection of a communication link when in the package. When the magnet is no longer sufficiently close to the magnetic sensing circuitry in the electronic device, such as when a lid that includes the magnet has been removed from the package and/or when the electronic device has been removed from the package, then the magnetic sensing circuitry may no longer control the power circuitry, and the communication circuitry may power on and wait for connection of a communication link to the electronic device.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

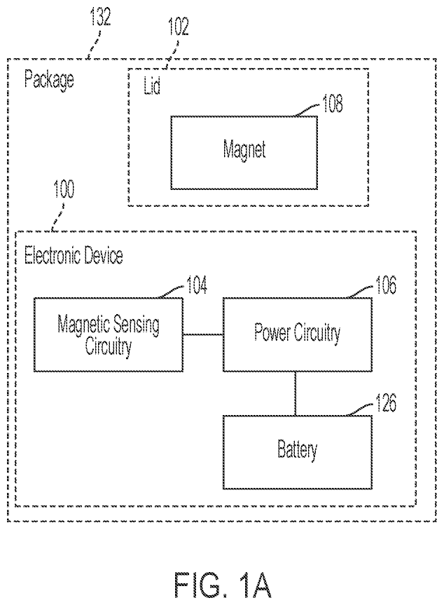

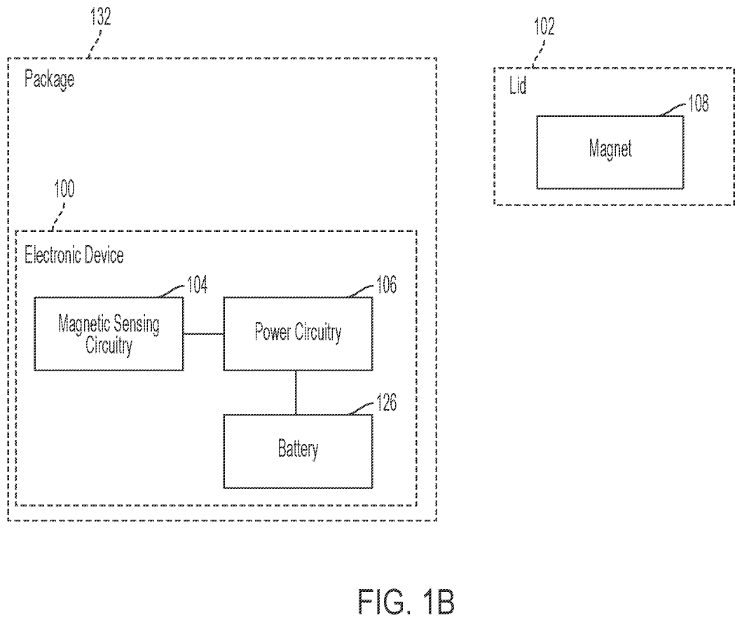

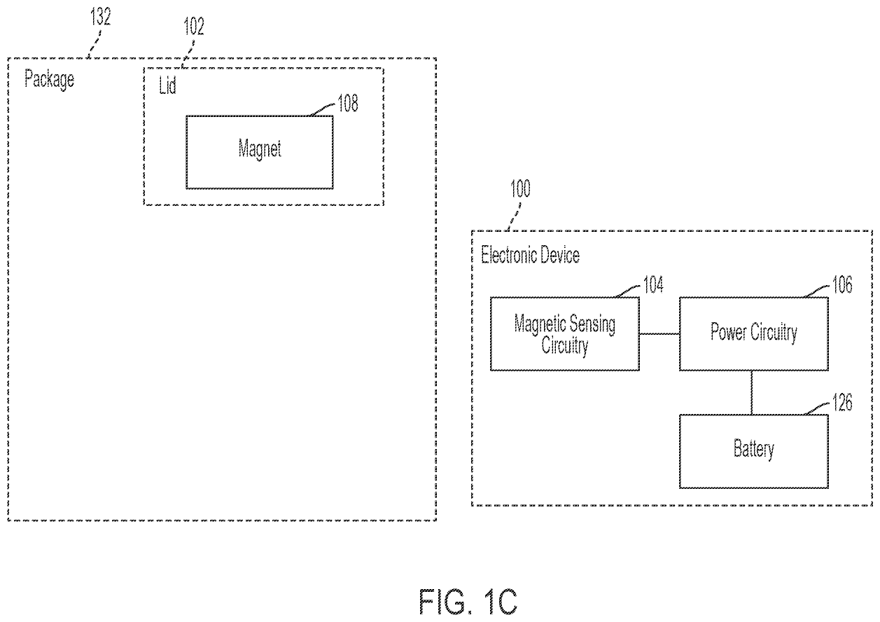

FIG. 1A is a schematic block diagram of a configuration of an example system for prolonging battery life in an electronic device, in accordance with certain embodiments described herein. FIG. 1A illustrates an electronic device 100, a package 132, and a lid 102. The electronic device 100 includes magnetic sensing circuitry 104, power circuitry 106, and a battery 126. The lid 102 includes a magnet 108. Thus, the magnet 108 is external to (e.g., not disposed on or within) the electronic device 100. The electronic device 100 may be any type of electronic device. While the below description may use an ultrasound device as an example, other electronic devices, such as smartphones, tablets, laptops, etc., may also be used. In FIG. 1A, the lid 102 and the electronic device 100 are illustrated within the package 132 to indicate a configuration in which the electronic device 100 is within the package 132 and the lid 102 is on the package 132 so as to close the package 132.

The magnetic sensing circuitry 104 is coupled to the power circuitry 106. The power circuitry 106 is coupled to the battery 126. The magnetic sensing circuitry 104 may be any circuitry configured to provide an output based on a detected magnetic field. For example, the magnetic sensing circuitry 104 may be a digital-output magnetic sensor configured to detect the magnetic field of a magnet and provide a digital output signal based on the detection. As particular examples, the magnetic sensing circuitry 104 may be one of the following parts: TCS30SPU, TCS30NPU, TCS30DPU, TCS30DLU, TCS40DPR, or TCS40DLR, manufactured by Toshiba. The magnetic sensing circuitry 104 may be disposed sufficiently far enough away from magnetic influences (e.g., transformers) and magnetic shielding in the electronic device 100 such that the magnetic influences of the electronic device itself do not cause the magnetic sensing circuitry 104 to detect other magnetic fields besides the magnet 108, and such that the magnetic shielding does not negatively affect the ability of the magnetic sensing circuitry 104 to detect the magnet 108. Further description of the magnetic sensing circuitry 104 may be found below with reference to the magnetic sensing circuitry 504.

The power circuitry 106 may be any circuitry providing power from the battery 126 to the electronic device 100 or a portion thereof. In some embodiments, the magnetic sensing circuitry 104 may be configured to control the power circuitry 106 based on detecting a magnetic field. For example, the magnetic sensing circuitry 104 may be configured to disable one or more power rails in the power circuitry 106 that provide power from the battery 126 to the electronic device 100 or a portion thereof based on detecting a magnetic field.

The package 132 may be any package in which the electronic device 100 is shipped and/or stored. For example, the package 132 may be a box or a case. In the example embodiment, the lid 102 is the lid for the package 132 and includes the magnet 108. The magnet 108 may be disposed (e.g., embedded) in a portion of the lid 102 such that, when the electronic device 100 is in the package 132, the magnet 108 is adjacent to the portion of the electronic device 100 in which the magnetic sensing circuitry 104 is disposed.

In operation, the magnetic sensing circuitry 104 may disable one or more power rails in the power circuitry 106 that provide power to the electronic device 100 or a portion thereof upon detecting the magnetic field of the magnet 108. Thus, in the configuration of FIG. 1A, in which the electronic device 100 is in the package 132 and the lid 102 is on the package 132 so as to close the package 132, the magnet 108 of the lid 102 may be sufficiently close to the electronic device 100 such that the magnetic sensing circuitry 104 detects the magnetic field of the magnet 108 and disables one or more power rails in the power circuitry 106 that provide power to the electronic device 100 or a portion thereof. It may be appropriate to power down the electronic device 100 or a portion thereof because when the electronic device 100 is in the package 132, the electronic device 100 may not be ready for use. When the magnetic sensing circuitry 104 keeps the electronic device 100 or a portion thereof powered down, current draw from battery 126 may be reduced, and the life of the battery 126 may be prolonged.

FIG. 1B is a schematic block diagram of another configuration of the example system of FIG. 1A for prolonging battery life in an electronic device, in accordance with certain embodiments described herein. In FIG. 1B, the electronic device 100 is illustrated within the package 132 but the lid 102 is illustrated external to the package 132 to indicate a configuration in which the electronic device 100 is within the package 132 but the lid 102 has been removed from the package 132 so as to open the package 132. Thus, in the configuration of FIG. 1B, the lid 102 may not be sufficiently close to the electronic device 100, such that the magnetic sensing circuitry 104 cannot detect the magnetic field of the magnet 108. When the magnetic sensing circuitry 104 cannot detect the magnetic field of the magnet 108, the magnetic sensing circuitry 104 may not control the power circuitry 106. The power circuitry 106 may then enter a default state in which the power rails in the power circuitry 106 provide power to the electronic device 100 or a portion thereof. It may be appropriate to power on the electronic device 100 or a portion thereof because when the electronic device 100 is not in the package 132 and/or the electronic device 100 is in the package 132 but lid 102 is not on the package 132, the electronic device 100 may be ready for use. In some embodiments, multiple powered states may be available, and when the magnetic sensing circuitry 104 cannot detect the magnetic field of the magnet 108 the power circuitry 106 may enter one of multiple potential power states. For example, the power circuitry 106 may enter a low power state that is higher power than the state in which magnetic sensing circuitry 104 detects the magnetic field of magnet 108, or may enter a higher powered state. In such embodiments, which powered state is entered may depend on factors other than whether the magnetic field of the magnet 108 is detected, such as whether the electronic device is in active use or not.

FIG. 1C is a schematic block diagram of another configuration of the example system of FIG. 1A for prolonging battery life in an electronic device, in accordance with certain embodiments described herein. In FIG. 1C, the electronic device 100 is illustrated external to the package 132 to indicate a configuration in which the electronic device 100 is removed from the package 132, such that the magnetic sensing circuitry 104 cannot detect the magnetic field of the magnet 108. The lid 102 is illustrated within the package 132 to indicate that the lid 102 is on the package 132 so as to close the package 132. However, the lid 102 may also be anywhere that is sufficiently removed from the electronic device 100 such that the magnetic sensing circuitry 104 cannot detect the magnetic field of the magnet 108. Further description of the situation when the magnetic sensing circuitry 104 cannot detect the magnetic field of the magnet 108 may be found with reference to FIG. 1B.

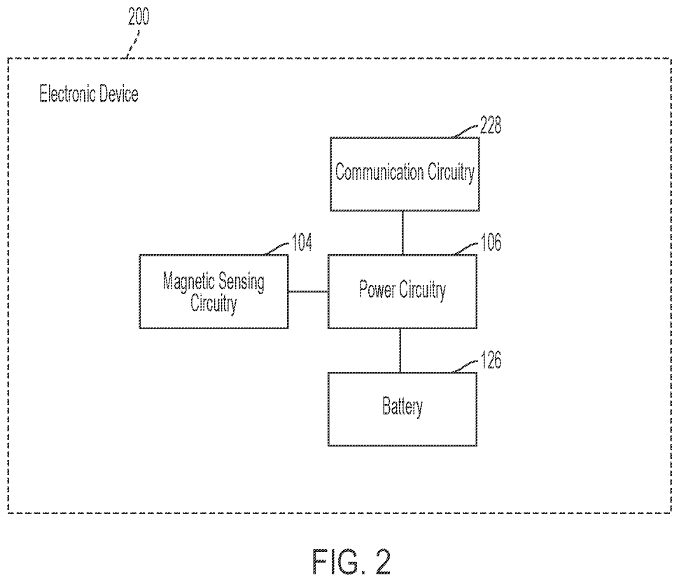

FIG. 2 is a schematic block diagram of an example electronic device 200, in accordance with certain embodiments described herein. The electronic device 200 may be the same as the electronic device 100, but with communication circuitry 228 of the electronic device 200 explicitly illustrated. The communication circuitry 228 may be configured to facilitate communication between the electronic device 200 and a processing device. The processing device may be, for example, a mobile phone, tablet, or a laptop. The communication circuitry 228 may be wired communication circuitry for communicating over a wired communication link (e.g., over Ethernet, a Universal Serial Bus (USB) cable or a Lightning cable) or wireless communication circuitry for communicating over a wireless communication link (e.g., over a BLUETOOTH, WiFi, or ZIGBEE wireless communication link).

The electronic device 200 may have a low power state in which the communication circuitry 228 may be powered on but other circuitry in the electronic device 200 may be powered off. When the communication circuitry 228 detects connection of a communication link (e.g., a cable) between the electronic device 200 and the processing device, the communication circuitry 228 may be configured to turn on other circuitry in the electronic device 200. In some embodiments, the communication circuitry 228 may be configured to receive or not receive power from the power circuitry 104 based on whether the magnetic sensing circuitry 104 detects or does not detect a magnetic field (e.g., of the magnet 108). For example, when the magnetic sensing circuitry 104 detects a magnetic field (e.g., of the magnet 108), the magnetic sensing circuitry 104 may be configured to disable one or more power rails in the power circuitry 106 that provide power to the communication circuitry 228. When the communication circuitry 228 has been powered down, the communication circuitry 228 may be unable to turn on other circuitry in the electronic device 200 upon detecting connection of a communication link. Additionally, when the communication circuitry 228 has been powered down, the electronic device 200 may draw less current from the battery 126 than when the communication circuitry 228 has been enabled. When the magnetic sensing circuitry 104 cannot detect a magnetic field, the magnetic sensing circuitry 104 may be configured to not control the power circuitry 106. The power circuitry 106 may then be configured to enter a default state in which the power rails in the power circuitry 106 provide power to the communication circuitry 228.

Thus, as an example, when the electronic device 200 is in the package 132 and the lid 102 is on the package 132, the magnet 108 of the lid 102 may be sufficiently close to the electronic device 200 such that the magnetic sensing circuitry 104 detects the magnetic field of the magnet 108. The communication circuitry 228 may not receive power from the power circuitry 106 when the magnetic sensing circuitry 104 detects the magnetic field of the magnet 108. For example, the magnetic sensing circuitry 104 may disable one or more power rails in the power circuitry 106 that provide power to the communication circuitry 228. It may be appropriate to power down the communication circuitry 228 because the electronic device 200 may be in the package 132 and not be ready for connection of a communication link (e.g., not be ready for connection of a wired cable). When the magnetic sensing circuitry 104 keeps the communication circuitry 228 powered down, current draw from the battery 126 may be reduced, and the life of the battery 126 may be prolonged. When the lid 102 is not sufficiently close to the electronic device 200, such that the magnetic sensing circuitry 104 cannot detect the magnetic field of the magnet 108 (e.g., the lid 102 has been removed from the package 132 and/or the electronic device 200 has been removed from the package 132), the magnetic sensing circuitry 104 may not control the power circuitry 106. The power circuitry 106 may then enter a default state in which the power rails in the power circuitry 106 provide power to the communication circuitry 228. It may be appropriate to power on the communication circuitry 228 because when the electronic device 200 is not in the package 132 and/or the electronic device 200 is in the package 132 but the lid 102 is not on the package 132, the electronic device 200 may be ready for connection of a communication link (e.g., ready for connection of a wired cable).

As an example, in some embodiments the electronic device 200 may be an ultrasound device configured to be in operative communication with a processing device such as a smartphone or a tablet. The processing device may be configured to transmit configuration commands to the ultrasound device, and the ultrasound device may be configured to collect ultrasound data and transmit the ultrasound data to the processing device for display as an ultrasound image. The communication circuitry may be a USB subsystem facilitating communication between the ultrasound device and the processing device. The ultrasound device may have a low power state in which the USB subsystem may be on but other circuitry in the ultrasound device may be off. When the USB subsystem detects connection of a USB cable to the ultrasound device, the USB subsystem may be configured to turn on other circuitry in the ultrasound device so that the ultrasound device may begin to collect data. The ultrasound device may be shipped in the package 132 such that the magnet 108 in the lid 102 of the package 132 causes the magnetic sensing circuitry 104 in the ultrasound device to disable power rails to the USB subsystem. This may be helpful in reducing power consumption by the USB subsystem, and it may be appropriate to power down the USB subsystem because a USB cable is unlikely to be connected to the ultrasound device when the ultrasound device is in the package 132 and the lid 102 is on the package 132. In other words, it may be unnecessary to keep the USB subsystem powered on and ready to detect connection of a USB cable when the ultrasound device is in the package 132 and the lid 102 is on the package 132.

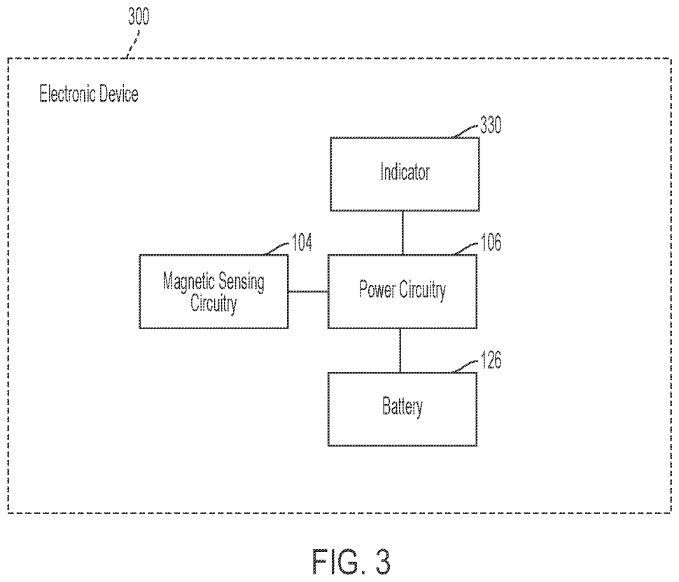

FIG. 3 is another schematic block diagram of an example electronic device 300, in accordance with certain embodiments described herein. The electronic device 300 may be the same as the electronic device 100, but with an indicator 330 of the electronic device 300 explicitly illustrated. The indicator 330 may include, for example, one or more light-emitting diodes (LEDs) on the exterior of the electronic device 300. In some embodiments, the indicator 330 may be configured to indicate a power level of the battery 126. For example, the indicator 330 may include multiple LEDs, and the number of LEDs that are on may indicative of the power level (e.g., the percentage to which the battery 126 is charged) of the battery 126. In some embodiments, the indicator 330 may indicate the power level of the battery 126 for a time period after a user presses a button on the exterior of the electronic device 300, and otherwise the indicator 330 may be off. In some embodiments, the indicator 330 may be configured to enter a turn-on sequence when the indicator 330 receives power from the power circuitry 106 after having not received power from the power circuitry 106 previously. The turn-on sequence may include, for example, one or more LEDs of the indicator 330 turning on and/or off in a particular timing sequence. In some embodiments, the turn-on sequence may include one or more LEDs of the indicator 330 turning on and staying on until the indicator 330 stops receiving power from the power circuitry 106.

In some embodiments, the indicator 330 may be configured to receive or not receive power from the power circuitry 104 based on whether the magnetic sensing circuitry 104 detects or does not detect a magnetic field (e.g., of the magnet 108). For example, when the magnetic sensing circuitry 104 detects a magnetic field (e.g., of the magnet 108), the magnetic sensing circuitry 104 may be configured to disable one or more power rails in the power circuitry 106 that provide power to the indicator 330. When the magnetic sensing circuitry 104 cannot detect a magnetic field, the magnetic sensing circuitry 104 may not control the power circuitry 106. The power circuitry 106 may then enter a default state in which the power rails in the power circuitry 106 provide power to the indicator 330. As another example, when the magnetic sensing circuitry 104 detects a magnetic field (e.g., of the magnet 108), the magnetic sensing circuitry 104 may be configured to disable one or more power rails in the power circuitry 106 that provide power to a portion of the electronic device 300 (e.g., a certain subsystem). When that portion of the electronic device 300 is not receiving power from the power circuitry 106, that portion of the electronic device 300 may be configured to keep the indicator 330 from receiving power from the power circuitry 106 as well. When the magnetic sensing circuitry 104 cannot detect a magnetic field, the magnetic sensing circuitry 104 may not control the power circuitry 106. The power circuitry 106 may then be configured to enter a default state in which the power rails in the power circuitry 106 provide power to the portion of the electronic device 300. Upon receiving power from the power circuitry 106, that portion of the electronic device 300 may be configured to enable the indicator 330 to receive power from the power circuitry 106.

Thus, as an example, when the electronic device 300 is in the package 132 and the lid 102 is on the package 132, the magnet 108 of the lid 102 may be sufficiently close to the electronic device 300 such that the magnetic sensing circuitry 104 detects the magnetic field of the magnet 108. The indicator 330 may be off when the magnetic sensing circuitry 104 detects the magnetic field of the magnet 108. For example, the magnetic sensing circuitry 104 may disable one or more power rails in the power circuitry 106 that provide power to the indicator 330. As another example, the magnetic sensing circuitry 104 may disable one or more power rails in the power circuitry 106 that provide power to a portion of the electronic device 300 (e.g., a certain subsystem), and that portion of the electronic device 300 may keep the indicator 330 from receiving power from the power circuitry 106 as well. When the lid 102 is not sufficiently close to the electronic device 300, such that the magnetic sensing circuitry 104 cannot detect the magnetic field of the magnet 108 (e.g., the lid 102 has been removed from the package 132), the magnetic sensing circuitry 104 may not control the power circuitry 106. The power circuitry 106 may then enter a default state. As one example, upon entering the default state, the power rails in the power circuitry 106 may provide power to the indicator 330. As another example, upon entering the default state, the power rails in the power circuitry 106 may provide power to a portion of the electronic device 300 (e.g., a subsystem), and upon receiving power, that portion of the electronic device 300 may enable the indicator 330 to receive power from the power circuitry 106. In any case, when the lid has been removed from the package 132, the indicator 330 may begin to receive power from the power circuitry 106, and therefore enter a turn-on sequence. This may provide a pleasing unboxing experience to a user.

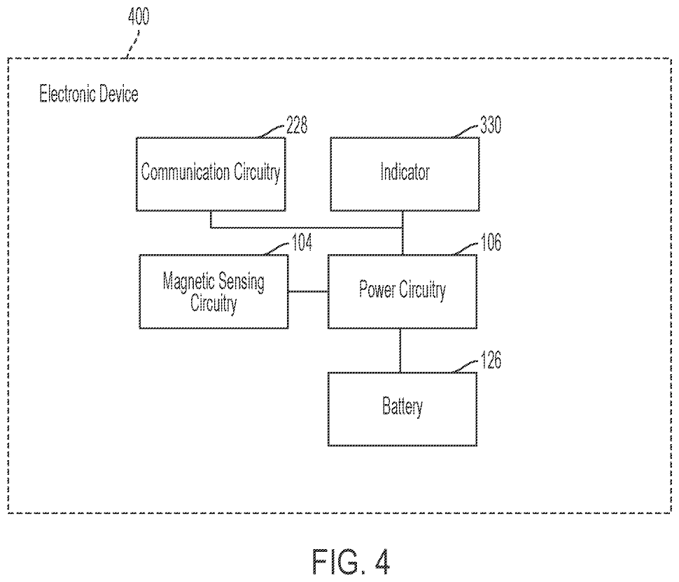

FIG. 4 is another schematic block diagram of an example electronic device 400, in accordance with certain embodiments described herein. The electronic device 400 may be the same as the electronic device 100, but with the communication circuitry 228 and the indicator 330 of the electronic device 400 explicitly illustrated. In some embodiments, the indicator 330 may be configured to receive or not receive power from the power circuitry 104 based on whether the magnetic sensing circuitry 104 detects or does not detect a magnetic field (e.g., of the magnet 108). For example, when the magnetic sensing circuitry 104 detects a magnetic field (e.g., of the magnet 108), the magnetic sensing circuitry 104 may be configured to disable one or more power rails in the power circuitry 106 that provide power to the communication circuitry 228 and the indicator 330. When the magnetic sensing circuitry 104 cannot detect a magnetic field, the magnetic sensing circuitry 104 may not control the power circuitry 106. The power circuitry 106 may then enter a default state in which the power rails in the power circuitry 106 provide power to the communication circuitry 228 and the indicator 330. As another example, when the magnetic sensing circuitry 104 detects a magnetic field (e.g., of the magnet 108), the magnetic sensing circuitry 104 may be configured to disable one or more power rails in the power circuitry 106 that provide power to the communication circuitry 228. When the communication circuitry 228 is not receiving power from the power circuitry 106, the communication circuitry 228 may be configured to keep the indicator 330 from receiving power from the power circuitry 106 as well. When the magnetic sensing circuitry 104 cannot detect a magnetic field, the magnetic sensing circuitry 104 may not control the power circuitry 106. The power circuitry 106 may then enter a default state in which the power rails in the power circuitry 106 provide power to the communication circuitry 228. Upon receiving power from the power circuitry 106, the communication circuitry 228 may be configured to enable the indicator 330 to receive power from the power circuitry 106.

Thus, as an example, when the electronic device 400 is in the package 132 and the lid 102 is on the package 132, the magnet 108 of the lid 102 may be sufficiently close to the electronic device 400 such that the magnetic sensing circuitry 104 detects the magnetic field of the magnet 108 and disables one or more power rails in the power circuitry 106 that provide power to the communication circuitry 528 and the indicator 330. It may be appropriate to power down the communication circuitry 228 because the electronic device 400 may be in the package 132 and not be ready for connection of a communication link (e.g., not be ready for connection of a wired cable). When the magnetic sensing circuitry 104 keeps the communication circuitry 228 powered down, current draw from the battery 126 may be reduced, and the life of the battery 126 may be prolonged. When the lid 102 is not sufficiently close to the electronic device 400, such that the magnetic sensing circuitry 104 cannot detect the magnetic field of the magnet 108 (e.g., the lid 102 has been removed from the package 132), the magnetic sensing circuitry 104 may not control the power circuitry 106. The power circuitry 106 may then enter a default state. As one example, in the default state, the power rails in the power circuitry 106 may provide power to the communication circuitry 228 and to the indicator 330. As another example, in the default state, the power rails in the power circuitry 106 may provide power to the communication circuitry 228, and upon receiving power from the power circuitry 106, the communication circuitry 228 may enable the indicator 330 to receive power from the power circuitry 106. It may be appropriate to power on the communication circuitry 228 because when the electronic device 400 is not in the package 132 and/or the electronic device 400 is in the package 132 but lid 102 is not on the package 132, the electronic device 400 may be ready for connection of a communication link (e.g., ready for connection of a wired cable). Additionally, when the lid 102 is removed from the package 132, the indicator 230 may enter a turn-on sequence, which may provide a pleasing unboxing experience to a user and indicate to the user that the communication circuitry 228 is ready to detect connection of a communication link and turn on the rest of the electronic device 400.

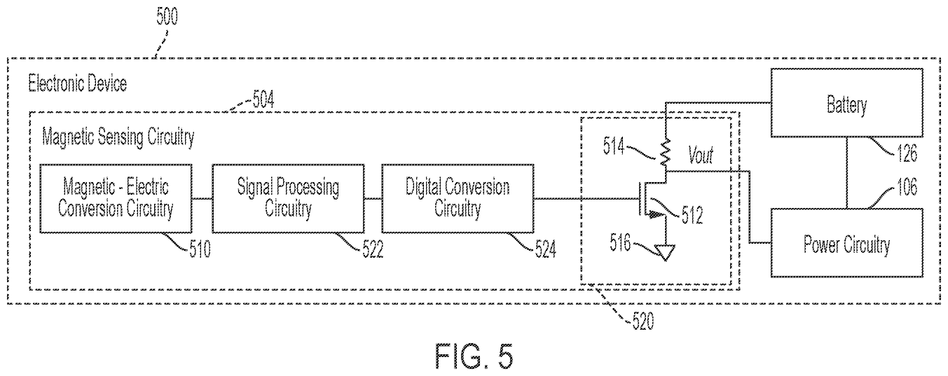

FIG. 5 is another schematic block diagram of an example electronic device 500, in accordance with certain embodiments described herein. The electronic device 500 includes magnetic sensing circuitry 504. The magnetic sensing circuitry 504 may be the same as the magnetic sensing circuitry 104, illustrated in more detail. The magnetic sensing circuitry 504 includes magnetic-electric conversion circuitry 510, signal processing circuitry 522, digital conversion circuitry 524, and output circuitry 520. The output circuitry 520 includes a transistor 512, a pull-up resistor 514, and ground 516. The output of the magnetic-electric conversion circuitry 510 is coupled to the input of the signal processing circuitry 522. The output of the signal processing circuitry 522 is coupled to the output of the digital conversion circuitry 524. The output of the digital conversion circuitry 524 is coupled to the output circuitry 520. In the output circuitry 520, the transistor 512 is a metal-oxide-semiconductor field-effect transistor (MOSFET), although another type of transistor such a bipolar junction transistor (BJT) may also be used. The gate of the transistor 512 is coupled to the output of the digital conversion circuitry 524. The drain of the transistor 512 (the voltage of which will be referred to as Vout, the digital output voltage of the magnetic sensing circuitry 504) is coupled to one terminal of the pull-up resistor 514, the other terminal of which is coupled to the battery 526. The source of the transistor 512 is coupled to ground.

The magnetic-electric conversion circuitry 510 may include a Hall effect sensor configured to output an electric voltage proportional to the strength of the magnetic field through the sensor. The signal processing circuitry 522 may be configured to process the output voltage from the magnetic-electric conversion circuitry 510. For example, the signal processing circuitry 522 may be configured to amplify the output voltage and/or cancel an offset in the output voltage. The digital conversion circuitry 524 may be configured to convert an analog voltage output from the signal processing circuitry 522 to a digital voltage. For example, the digital conversion circuitry 524 may include a comparator configured to compare the analog voltage output to a threshold voltage, and output a digital high voltage if the analog voltage output exceeds the threshold voltage and output a digital low voltage if the analog voltage output does not exceed the threshold voltage. Thus, the digital conversion circuitry 524 may be configured to output the digital low voltage when the detected magnetic field does not exceed a threshold, and may be configured to output the digital high voltage when the detected magnetic field does exceed the threshold.

The output circuitry 520 may be considered an open drain configuration. In operation, when the digital conversion circuitry 524 outputs a digital low voltage, the transistor 512 is at high impedance, and the pull-up resistor 514 pulls Vout to the voltage of the battery 526. In other words, Vout may be a digital high voltage. When the digital conversion circuitry 524 outputs a digital high voltage, the 0 512 is on and pulls Vout low towards ground 516. In other words, Vout may be a digital low voltage. It should be appreciated that other types of configuration for the output circuitry 520, such as a push-pull configuration, may be used.

The power circuitry 106 may be configured to receive Vout at a terminal that controls enabling of one or more power rails in the power circuitry 106 that provide power from the battery 126 to the electronic device 500 or a portion thereof (e.g., the communication circuitry 228 and/or the indicator 330, which are not illustrated in FIG. 5). When Vout is the digital low voltage (corresponding to the detected magnetic field exceeding a threshold level), the power circuitry 106 may be configured to disable the power rails. When Vout is the digital high voltage (corresponding to the detected magnetic field not exceeding a threshold level), the power circuitry 106 may be configured to enable the power rails.

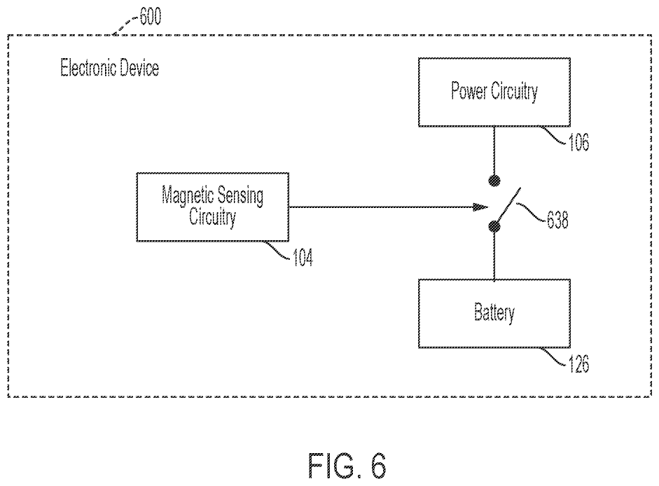

FIG. 6 is a schematic block diagram of another example system for prolonging battery life in an electronic device, in accordance with certain embodiments described herein. FIG. 6 includes an electronic device 600. The electronic device 600 includes a switch 638 between the battery 126 and the power circuitry 106 that is controlled by the magnetic sensing circuitry 104. Thus, rather than the magnetic sensing circuitry 104 controlling the power circuitry 106 (e.g., by disabling power rails in the power circuitry 106), the magnetic sensing circuitry 104 may be configured to control whether power circuitry 106 receives power from the battery 126 by coupling or decoupling the power circuitry 106 from the battery 126 based on whether the magnetic sensing circuitry 104 detects a magnetic field. This may help to reduce current draw from the battery 126 by decreasing the quiescent power used by the power circuitry 106. It should be appreciated that there may be multiple switches 638 between the power circuitry 106 and the battery 126 controlled by the magnetic sensing circuitry 104. It should also be appreciated that the switch 638 may be any type of circuit configured as a gate between the battery 126 and the power circuitry 106. The gating scheme of FIG. 6 may be implemented in any of the electronic devices described herein (such as the electronic devices 100, 200, 300, 400, 500, or 800).

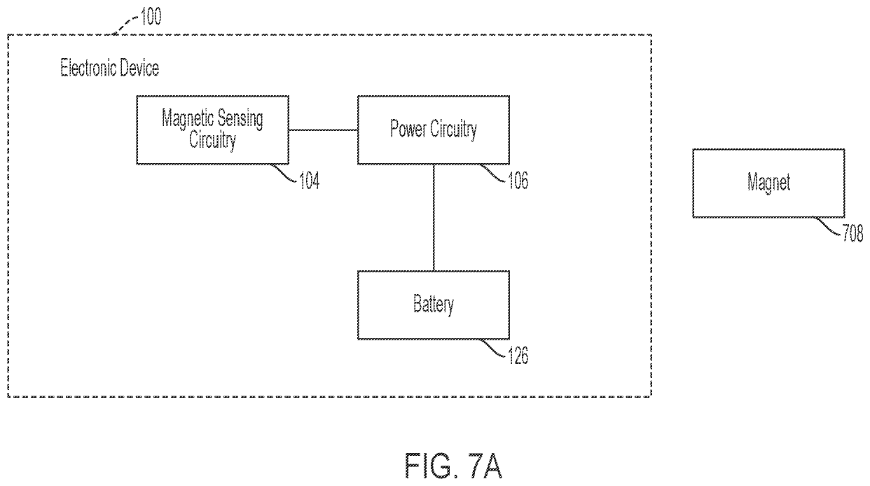

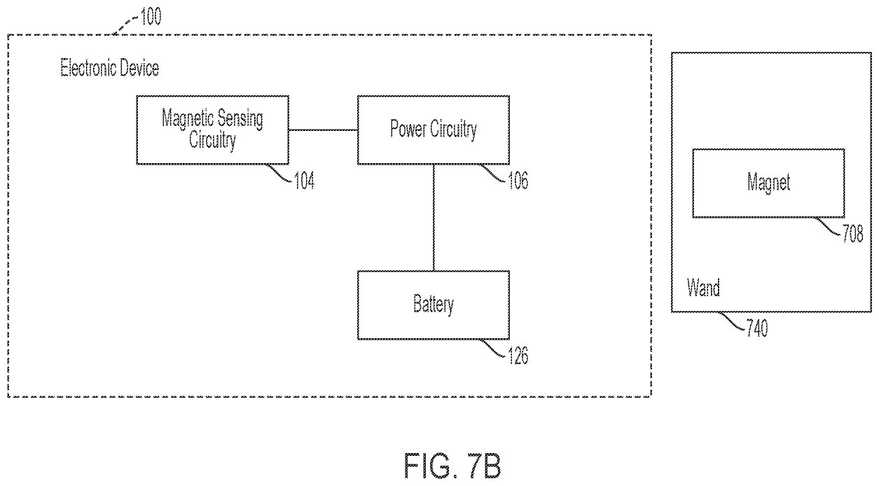

FIG. 7A is a schematic block diagram of another example system for prolonging battery life in the electronic device 100, in accordance with certain embodiments described herein. FIG. 7A includes the electronic device 100 and a magnet 708. In contrast to the magnet 108, the magnet 708 may not be included in a package. The magnet 708 may be any magnet that is external to (e.g., not disposed on or within) the electronic device 100. The magnet 708 may be a standalone magnet, or included in another object such as a wand. The electronic device 100 may be the same as the electronic device 200, 300, 400, 500, 600, or 800.

In operation, the magnetic sensing circuitry 104 may be configured to disable one or more power rails in the power circuitry 106 that provide power to the electronic device 100 or a portion thereof, upon detecting the magnetic field of the magnet 708. Thus, when the magnet 708 is positioned sufficiently close to the electronic device 100 such that the magnetic sensing circuitry 104 detects the magnetic field of the magnet 708, the magnetic sensing circuitry 104 may disable one or more power rails in the power circuitry 106 that provide power to the electronic device 100 or a portion thereof (e.g., the communication circuitry 228, not shown in figure). For example, a user may position the magnet 708 (e.g., position a wand containing the magnet 708) sufficiently close to the electronic device 100. When the magnet 708 is removed from being positioned sufficiently close to the electronic device 100 such that the magnetic sensing circuitry 104 cannot detect the magnetic field of the magnet 708, the power circuitry 106 may then enter a default state in which the power rails in the power circuitry 106 provide power to the electronic device 100 or a portion thereof (e.g., the communication circuitry 228). Disabling and then enabling the power rails in the power circuitry 106 may reset (e.g., restart the power sequence of) certain systems in the electronic device 100 (e.g., the communication circuitry 228).

FIG. 7B is a schematic block diagram of another example system for prolonging battery life in the electronic device 100, in accordance with certain embodiments described herein. FIG. 7B includes the electronic device 100 and a wand 740 that includes the magnet 708. In some embodiments, the wand may be any object that can be held over the electronic device such that the magnetic sensing circuitry 104 can detect the magnetic field of the magnet 708. The electronic device 100 may be the same as the electronic device 200, 300, 400, 500, 600, or 800.

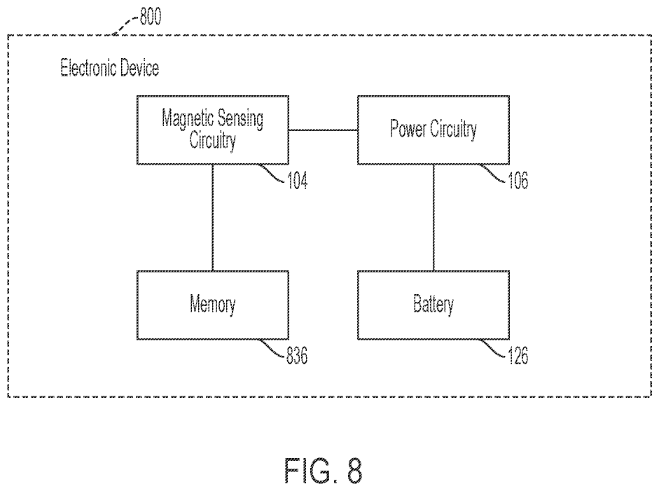

FIG. 8 is another schematic block diagram of an example electronic device 800, in accordance with certain embodiments described herein. The electronic device 800 includes memory 836. The electronic device 800 may be the same as the electronic devices 100, 200, 300, 400, 500, or 600 but with the memory 836 explicitly illustrated. In some embodiments, the memory 836 may be configured to store a value corresponding to the number of times that the magnetic sensing circuitry 104 may power down portions of the electronic device 800, for example by disabling power rails, before the magnetic sensing circuitry 104 is disabled from powering down portions of the electronic device 800 anymore. In some embodiments, the memory 836 may be configured to store a value for the number of times within a given time period that the magnetic sensing circuitry 104 may power down portions of the electronic device 800, for example by disabling power rails, before the magnetic sensing circuitry 104 is disabled from powering down portions of the electronic device 800 anymore. For example, the value stored in the memory 836 may be 1 prior to placement of the electronic device 800 in the package 132. Thus, the magnetic sensing circuitry 104 maybe disabled the first time the magnetic sensing circuitry 104 ceases to detect a magnetic field (e.g., of the magnet 108 or 708), such as when the electronic device 800 is first removed from the package 132. This may be helpful in preventing accidental activation of the magnetic sensing circuitry 104, and powering down of certain portions of the electronic device 800, during normal use of the electronic device 800 subsequent to removal of the electronic device 800 from the package 132. In some embodiments, the value stored in the memory 836 may be set to 1 (or a different number) prior to packaging and after certain portions of the electronic device have been reset (e.g., multiple times) by activating and deactivating the magnetic sensing circuitry 104 during factory testing. A standalone magnet such as the magnet 708 may be used for the activation and deactivation during factory testing. During this factory testing, the value stored in the memory 836 may be larger than 1, to allow for multiple reset cycles. In some embodiments, the memory 836 itself may control disabling of the magnetic sensing circuitry 104 based on the value stored in the memory 836. In some embodiments, control circuitry (not illustrated in the figure) may retrieve the value from the memory 836 and control disabling of the magnetic sensing circuitry 104 based on the value stored in the memory 836.

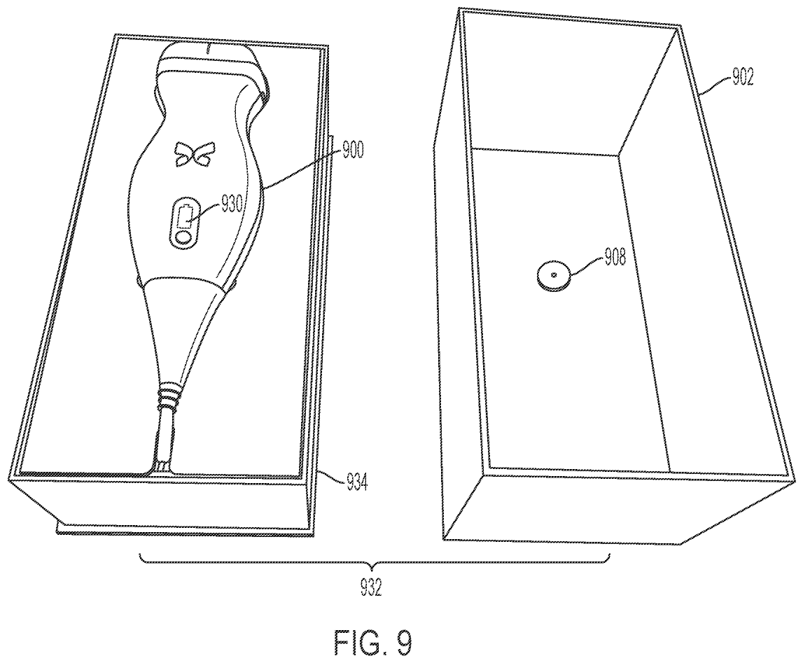

FIG. 9 is a perspective view of a system for prolonging battery life, in accordance with certain embodiments described herein. FIG. 9 illustrates an electronic device 900 and a package 932. The electronic device 900 includes an indicator 930. The package 932 includes a lid 902, a bottom portion 934, and a magnet 908. The indicator 930 is disposed on the exterior of the electronic device 900. The lid 902 is configured to fit over the bottom portion 934 of the package 932. The magnet 908 is disposed on an inner surface of the lid 902, such that when the lid 902 is placed over the bottom portion 934 of the package, the magnet 908 is adjacent to magnetic sensing circuitry in the electronic device 900. The electronic device 900 is disposed in the bottom portion 934 of the package 932. The electronic device 900 may be the same as the electronic devices 100, 200, 300, 400, 500, 600, or 800. The indicator 930 may be the same as the indicator 330. The package 932 may be the same as the package 132. The lid 902 may be the same as the lid 102. The magnet 908 may be the same as the magnet 108.

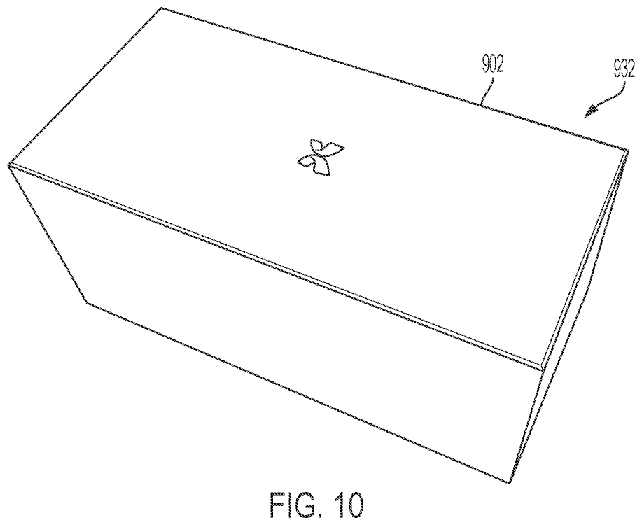

FIGS. 10-16 are perspective views of the system for prolonging battery life of FIG. 9 in operation, in accordance with certain embodiments described herein. Each successive figure illustrates a state of the system a time after the previous figure.

FIG. 10 illustrates the package 932 with the lid 902 placed over the bottom portion 934 of the package 932. The bottom portion 934 of the package 932 and the electronic device 900 are therefore not visible in FIG. 10.

When the magnetic sensing circuitry in the electronic device detects the magnetic field of the magnet 908 (e.g., when the electronic device 900 is in the bottom portion 934 of the package 932 and the lid 902 is placed on the bottom portion 934 of the package, as in FIG. 10), the indicator 930 may be powered down.

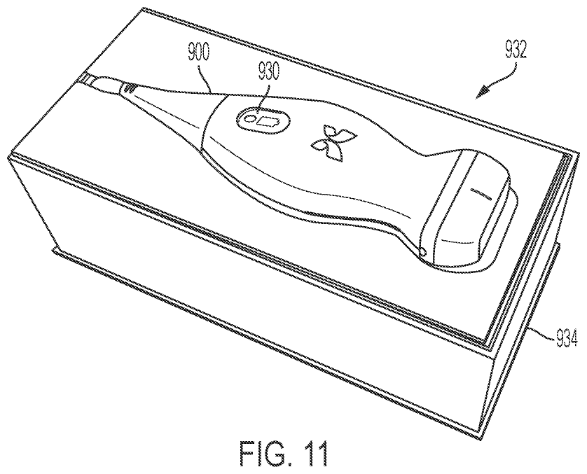

FIG. 11 illustrates the package 932 with the lid 902 removed from the bottom portion 934 of the package 932, thus revealing the package 932 and the electronic device 900. Based on magnetic sensing circuitry in the electronic device 100 no longer detecting the magnetic field of the magnet 908 disposed on the lid 902 (e.g., when the lid 902 is removed from the bottom portion 934 of the package 932), the indicator 930 may receive power, and a turn-on sequence for the indicator 930 may occur. The turn-on sequence may begin a time period after the indicator 930 initially receives power, in FIG. 11.

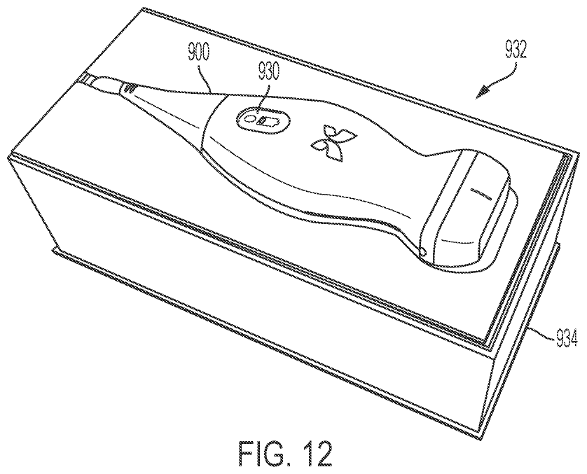

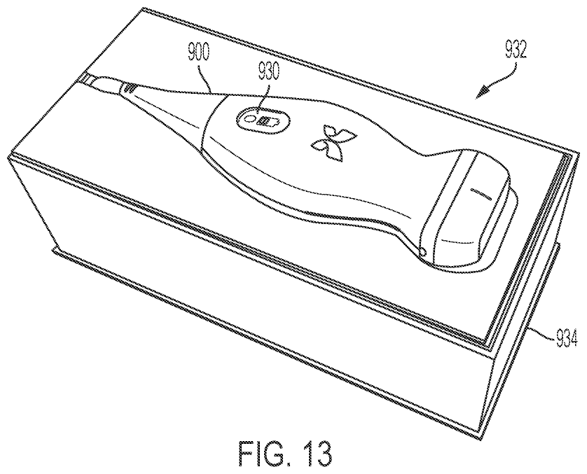

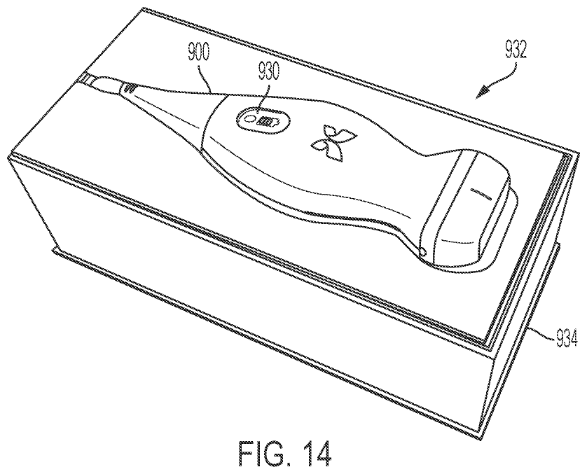

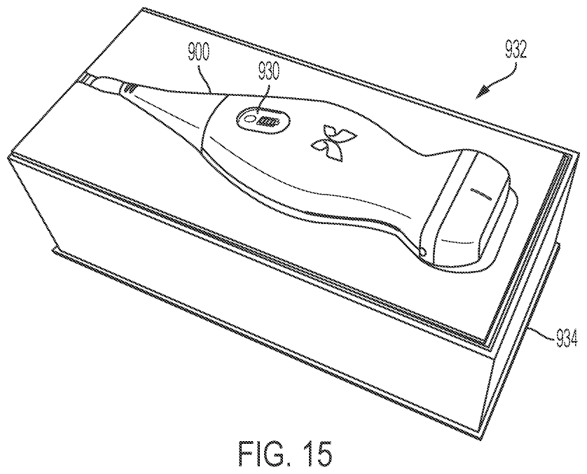

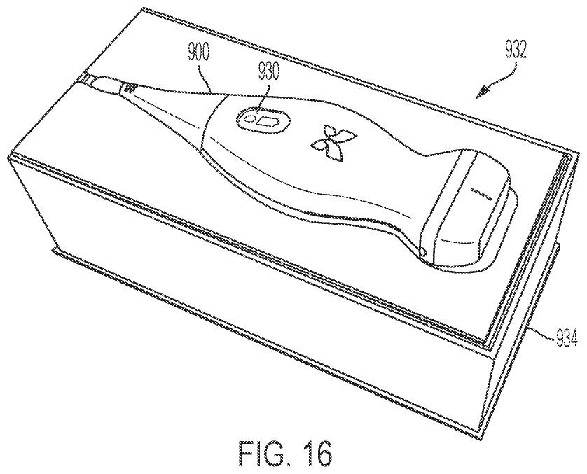

FIG. 12 illustrates that the turn-on sequence begins with a first LED of the indicator 930 turning on. FIG. 13 illustrates that the turn-on sequence continues with a second LED of the indicator 930 turning on. FIG. 14 illustrates that the turn-on sequence continues with a third LED of the indicator 930 turning on. FIG. 15 illustrates that the turn-on sequence continues with a fourth LED of the indicator 930 turning on. FIG. 16 illustrates that the turn-on sequence has concluded and the indicator 930 is off again. In other words, the indicator 930 turns off a time period after receiving power. It should be appreciated that other turn-on sequences are possible. For example, there may be more or fewer LEDs in the indicator 930, certain or all of the LEDs may turn on at the same time, certain or all of the LEDs may turn off at the same time, certain or all of the LEDs may blink a certain number of times, the indicator 930 may turn on right after receiving power, the indicator 930 may not turn off after the turn-on sequence, etc.

While the above description has described a magnet (e.g., the magnets 108 or 908) as disposed in a lid (e.g., the lids 102 or 902) of a package (e.g., the package 132 or 932), in some embodiments the magnet may be disposed in another portion of the package. Additionally, it should be appreciated that while the above description has described and illustrated packages that have removable lids, certain embodiments may have different types of packages. For example, a package may have a hinged lid that may be rotated but not removed from the package to open the package. When the package is open, the lid and the magnet disposed on or in the lid may be sufficiently far from the electronic device in the package such that magnetic sensing circuitry in the electronic device can no longer detect the magnetic field of the magnet. Some embodiments include providing an electronic device (e.g., the electronic devices 100, 200, 300, 400, 500, 600, 800, or 900) including magnetic sensing circuitry (e.g., the magnetic sensing circuitry 104 or 504) configured to control power received by a portion of the electronic device based on detecting a magnet that is external to the electronic device. Some embodiments include providing such an electronic device packaged in a package (e.g., the packages 132 or 932) including a magnet (e.g., the magnets 108 or 908) to a customer. For example, some embodiments include shipping the electronic device packaged in the package to a customer. Some embodiments include providing such an electronic device along with a magnet (e.g., the magnet 708) that may be standalone or included in some other object.

Various inventive concepts may be embodied as one or more processes, of which examples have been provided. The acts performed as part of each process may be ordered in any suitable way. Thus, embodiments may be constructed in which acts are performed in an order different than illustrated, which may include performing some acts simultaneously, even though shown as sequential acts in illustrative embodiments. Further, one or more of the processes may be combined and/or omitted, and one or more of the processes may include additional steps.

Various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

As used herein, reference to a numerical value being between two endpoints should be understood to encompass the situation in which the numerical value can assume either of the endpoints. For example, stating that a characteristic has a value between A and B, or between approximately A and B, should be understood to mean that the indicated range is inclusive of the endpoints A and B unless otherwise noted.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. A system, comprising:
an ultrasound device comprising magnetic sensing circuitry configured to control power received by a portion of the ultrasound device based on detecting a magnet that is external to the ultrasound device.

2. The system of claim 1, wherein the ultrasound device further comprises power circuitry, and wherein the magnetic sensing circuitry is configured to control the power circuitry based on detecting the magnet.

3. The system of claim 2, wherein the magnetic sensing circuitry is configured to disable one or more power rails in the power circuitry that provide power to the portion of the ultrasound device based on detecting the magnet.

4. The system of claim 2, wherein the magnetic sensing circuitry is configured to disable one or more power rails in the power circuitry when the magnetic sensing circuitry detects the magnet.

5. The system of claim 2, wherein the ultrasound device is configured such that, when the magnetic sensing circuitry cannot detect the magnet, the power circuitry is configured to enter a default state in which power rails in the power circuitry provide power to the portion of the ultrasound device.

6. The system of claim 1, wherein the ultrasound device further comprises power circuitry and a battery, and wherein the magnetic sensing circuitry is configured to control a coupling between the power circuitry and the battery based on whether the magnet is detected.

7. The system of claim 6, wherein the magnetic sensing circuitry is configured to decouple the power circuitry from the battery when the magnetic sensing circuitry detects the magnet.

8. The system of claim 6, wherein the ultrasound device is configured such that, when the magnetic sensing circuitry cannot detect the magnet, the power circuitry is configured to enter a default state in which the power circuitry is coupled to the battery.

9. The system of claim 1, wherein the magnetic sensing circuitry comprises a digital-output magnetic sensor.

10. The system of claim 1, wherein the ultrasound device further comprises communication circuitry, and the portion of the ultrasound device comprises the communication circuitry.

11. The system of claim 10, wherein the communication circuitry is configured to facilitate communication between the ultrasound device and a processing device, and the processing device comprises a mobile phone, a tablet, or a laptop.

12. The system of claim 10, wherein:
the ultrasound device has a low power state in which the communication circuitry is powered on but other circuitry in the ultrasound device is powered off; and
the communication circuitry is configured, based on detecting connection of a communication link between the ultrasound device and the processing device, to turn on the other circuitry in the ultrasound device.

13. The system of claim 1, wherein the ultrasound device further comprises an indicator, and the indicator is configured to receive or not receive power based on whether the magnetic sensing circuitry detects or does not detect the magnet.

14. The system of claim 13, wherein the indicator comprises a light-emitting diode (LED) on an exterior of the ultrasound device.

15. The system of claim 13, wherein the indicator is configured to enter a turn-on sequence upon receiving power.

16. The system of claim 13, further comprising a package comprising a lid, the lid comprising the magnet, and wherein:
the ultrasound device and the lid are configured such that, when the lid is removed from the package, the indicator enters a turn-on sequence.

17. The system of claim 1, further comprising a package that comprises the magnet.

18. The system of claim 17, wherein the package comprises a lid, and the lid comprises the magnet.

19. The system of claim 17, wherein the magnet is disposed in a portion of the package such that, when the ultrasound device is disposed in the package, the magnet is adjacent to a portion of the ultrasound device in which the magnetic sensing circuitry is disposed.

20. The system of claim 17, wherein the ultrasound device and the package are configured such that, when the ultrasound device is in the package, the magnet is sufficiently close to the ultrasound device such that the magnetic sensing circuitry detects the magnet.
